(19) United States
(12) Patent Application Publication
(10) Pub. No.: US 2017/0246245 A1
(43) Pub. Date: Aug. 31, 2017

(54) NOVEL PHARMACEUTICAL COMPOSITION FOR TREATING ALZHEIMER'S DISEASE

(71) Applicant: INDUSTRY-ACADEMIC COOPERATION FOUNDATION GYEONGSANG NATIONAL UNIVERSITY, Jinju-si, Gyeongsangnam-do (KR)

(72) Inventor: Myeong Ok KIM, Jinju-si, Gyeongsangnam-do (KR)

(21) Appl. No.: 15/510,826

(22) PCT Filed: Sep. 15, 2014
(86) PCT No.: PCT/KR2014/008578

§ 371 Date: Mar. 13, 2017

(51) Int. Cl. A61K 38/16; A61K 9/00

(52) U.S. Cl. CPC A61K 38/168; A61K 9/0053; A61K 9/0019

(57) ABSTRACT

The present invention relates to a pharmaceutical composition, and more specifically, to a novel pharmaceutical composition for treating Alzheimer's disease including osmotin as an active ingredient.

### TECHNICAL FIELD

The present invention relates to a pharmaceutical composition, and more specifically, to a pharmaceutical composition for treating Alzheimer's disease.

### BACKGROUND ART

A dementia disease basically entails short- and long-term memory impairments, which are also observed as the basic symptoms, and it is thought to consist of memory impairment, disorientation based on the same, and high-level brain function disorder.

Alzheimer's dementia (hereinafter, “AD”) is accompanied by various psychological and behavioral symptoms along with an abnormal progress of deterioration in mobility and cognitive ability. It begins with mild symptoms initially and eventually leads to such an extent that voluntary personal life is impossible and thus its effect is very serious. In patients with “AD”, a functional deterioration of neurotransmission in acetylcholine, glutamic acid, neuropeptides, and monoamine systems occurs and thus the functional disorder in these neurotransmission systems is presumed to be the main cause of “AD”. Additionally, from the aspect of neurocytotoxic activity induced by β-amyloid peptide, the pathogenesis by the elimination of hippocampal neurons through the extracellular accumulation of senile plaques of β-amyloid peptides is also presumed to be one of the main causes of “AD”.

Reviewing prior documents with respect to osmotin-related therapeutic compositions, Korean Registration Patent No. 1308232 discloses a composition for preventing and treating brain damage by alcohol comprising osmotin.

### DISCLOSURE OF THE INVENTION

### Technical Problem

However, although therapeutic agents for inhibiting the progress of Alzheimer's disease are being developed, an effective method for properly treating Alzheimer's disease has not yet been developed. In order to solve various problems including the above-mentioned problems, an object of the present invention is to provide a composition for preventing or treating Alzheimer's disease. However, these objects are for illustrative purposes and the present invention should not be limited by the same.

### Technical Solution

In an aspect of the present invention, there is provided a pharmaceutical composition for preventing or treating Alzheimer's disease, containing osmotin as an active ingredient.

In another aspect of the present invention, there is provided a health functional food for improving cognitive function and memory, containing osmotin as an active ingredient.

In still another aspect of the present invention, there is provided a method of treating Alzheimer's disease in a subject, including administering to the subject a therapeutically effective amount of osmotin.

In still another aspect of the present invention, there is provided a method of improving cognitive function and memory of a subject, including administering to the subject a therapeutically effective amount of osmotin.

### Advantageous Effects

According to an exemplary embodiment of the present invention constituted as described above, a pharmaceutical composition which can effective treat Alzheimer's disease and a health functional food which can improve cognitive and memory impairments including Alzheimer's disease and improve memory can be prepared. Of course, the scope of the present invention should not be limited by these effects.

### BRIEF DESCRIPTION OF THE DRAWINGS

FIG. 1 is a graph (upper part) illustrating the results of escape latency and moving routes in Morris Water Maze Test (lower part) according to an exemplary embodiment of the present invention.

FIG. 2 is a graph illustrating the results of Morris Water Maze Test according to an exemplary embodiment of the present invention.

FIG. 3 is a graph illustrating the results of the Y-maze spontaneous alternation test according to an exemplary embodiment of the present invention.

FIG. 4 is an image illustrating the results of western blotting analysis of marker proteins representing the synapse density in the hippocampus of a control mouse administered with vehicle only, a model mouse with Alzheimer's disease (APPsw) administered with vehicle only, and a model mouse with Alzheimer's disease administered with osmotin, according to an embodiment of the present invention.

FIG. 5 is an image illustrating the results of western blotting analysis of marker proteins relating to the formation of Aβ plaques in the hippocampus of the control mouse administered with vehicle only, a model mouse with Alzheimer's disease (APPsw) administered with vehicle only, and a model mouse with Alzheimer's disease administered with osmotin, according to an embodiment of the present invention.

FIG. 6 is an image illustrating the results of western blotting analysis with respect to Ap plaques in the cerebral cortex of a control mouse administered with vehicle only, a model mouse with Alzheimer's disease (APPsw) administered with vehicle only, and a model mouse with Alzheimer's disease administered with osmotin, according to an embodiment of the present invention.

FIG. 7 is an image illustrating the results of western blotting analysis of a series of proteins relating to tau phosphorylation in the hippocampus of a control mouse administered with vehicle only, a model mouse with Alzheimer's disease (APPsw) administered with vehicle only, and a model mouse with Alzheimer's disease administered with osmotin, according to an embodiment of the present invention.

FIG. 8 is an image illustrating the results of western blotting analysis confirming the level of tau phosphorylation in the cerebral cortex of a control mouse administered with vehicle only, a model mouse with Alzheimer's disease (APPsw) administered with vehicle only, and a model mouse with Alzheimer's disease administered with osmotin, according to an embodiment of the present invention.

FIG. 9 is a series of images of immunofluorescence microscope with regard to synaptophysin and GABA_{B1}R in the hippocampus of a control mouse administered with vehicle only, a model mouse with Alzheimer's disease (APPsw) administered with vehicle only, and a model mouse with Alzheimer's disease administered with osmotin, according to an embodiment of the present invention, wherein scale bars represent 200 μm and 10 μm, respectively.

FIG. 10 is a graph illustrating the quantitation of the fluorescent signals from the results of FIG. 9, wherein *P<0.05, **P<0.01, and ***P<0.001.

FIG. 11 is a series of images illustrating the results of immunofluorescent microscope with regard to NgR1 and GABA_{B1}R in the DG sector of the hippocampus of a control mouse administered with vehicle only, a model mouse with Alzheimer's disease (APPsw) administered with vehicle only, and a model mouse with Alzheimer's disease administered with osmotin, according to an embodiment of the present invention, wherein scale bars represent 200 μm and 10 μm, respectively.

FIG. 12 is a graph illustrating the quantitation of the fluorescent signals from the results of FIG. 11, wherein *P<0.05, **P<0.01, and ***P<0.001.

FIG. 13 is a series of images illustrating the results of immunofluorescent microscope with regard to NgR1 and GABA_{B1}R in the CA1 sector of the hippocampus of a control mouse administered with vehicle only, a model mouse with Alzheimer's disease (APPsw) administered with vehicle only, and a model mouse with Alzheimer's disease administered with osmotin, according to an embodiment of the present invention, wherein scale bars represent 200 μm and 10 μm, respectively.

FIG. 14 is a graph illustrating the quantitation of the fluorescent signals from the results of FIG. 13, wherein *P<0.05, **P<0.01, and ***P<0.001.

FIG. 15 is a series of images illustrating the results of thioflavin S staining of amyloid plaques and the immunofluorescent microscopic images of amyloid μ and in the hippocampus of a control mouse administered with vehicle only, a model mouse with Alzheimer's disease (APPsw) administered with vehicle only, and a model mouse with Alzheimer's disease administered with osmotin, according to an embodiment of the present invention, wherein scale bars represent 200 μm, 50 μm, and 10 μm, respectively.

FIG. 16 is a graph illustrating the quantitation of the fluorescent signals from the results of FIG. 15, wherein *P<0.05 and **P<0.01.

FIG. 17 is a series of immunofluorescence microscopic images illustrating the reduction in the formation of Aμ plaques in the cerebral cortex of a control mouse administered with vehicle only, a model mouse with Alzheimer's disease (APPsw) administered with vehicle only, and a model mouse with Alzheimer's disease administered with osmotin, according to an embodiment of the present invention.

FIG. 18 is a graph illustrating the quantitation of the fluorescent signals from the immunofluorescence analysis of FIG. 17.

FIG. 19 is a series of images illustrating the results of immunofluorescence analysis with regard to the measurement of the amount of phosphorylated tau (Ser⁴¹³) in the hippocampus (A) and the cerebral cortex (B) of a control mouse administered with vehicle only, a model mouse with Alzheimer's disease (APPsw) administered with vehicle only, and a model mouse with Alzheimer's disease administered with osmotin, according to an embodiment of the present invention.

FIG. 20 is a graph illustrating the quantitation of the immunofluorescent signals from the results of FIG. 17, wherein *P<0.05, **P<0.01, and ***P<0.001.

FIG. 21 is a series of graphs illustrating the analysis results of cell survival rates (A and B), cytotoxicity (C and D), and caspase-3/7 activities (E and F) of neurons in the hippocampus (A, C, and E) and cerebral cortex (B, D, and F) neurons in mice treated with various concentrations of osmotin, according to an embodiment of the present invention.

FIG. 22 is a schematic diagram summarizing the experimental results representing mechanism of osmotin in alleviating Alzheimer's symptoms, according to an embodiment of the present invention.

### MODE FOR CARRYING OUT THE INVENTION

### Definition of Terms

As used herein, “osmotin” is a natural protein isolated from a plant and it has a chemical structure similar to that of adiponectin, which is an animal hormone having the functions of lipolysis and inhibition of diabetes, and consists of about 150 to 205 amino acids depending on the subject. Osmotin (24 kDa) is a stable protein belonging to the pathogenesis-related protein (PR-5) family having a homology to thaumatin, which is a sweet-testing protein, and is known to induce intracellular signaling in yeasts. Additionally, a previous report revealed that osmotin has a similar function to that of proteins which are involved in the inhibition of obesity and diabetes in the body.

An aspect of the present invention provides a pharmaceutical composition for treating Alzheimer's disease containing osmotin as an active ingredient.

In the above pharmaceutical composition, the osmotin may be one isolated/purified from a plant, wherein the plant may be one that belongs to the genus Nicotiana. The osmotin may consist of an amino acid sequence represented by SEQ ID NO: 1, and it may be produced by genetic recombination technology using the cells of prokaryotes or eukaryotes such as yeasts, plants, insects, and mammals based on the above amino acid sequence.

The pharmaceutical composition may be administered orally or parenterally, and for parenteral administration, the composition may be administered intravenously, subcutaneously, intramuscularly, intraperitoneally, etc.

An appropriate dose of the pharmaceutical composition may vary depending on the method of formulations, method of administration, the age, body weight, gender, disease conditions of a patient, diet, administration time, administration route, excretion rate, reaction sensitivity, etc., and a skilled physician with a moderate level of experience can easily determine and prescribe an effective dose for the desired prevention or treatment. According to a preferred embodiment of the present invention, an appropriate daily dose is in a range of 100 μg/kg to 1 mg/kg (body weight). The pharmaceutical composition may be administered once daily or a few divided doses for several weeks.

Additionally, the pharmaceutical composition may be prepared in a unit dose form or prepared to be contained in a multi-dose container by formulating using a pharmaceutically acceptable carrier and/or excipient, according to a method that one of ordinary skill in the art to which the present invention pertains can easily perform. In particular, the formulation may be in the form of a solution in an oil or aqueous medium, a suspension, or an emulsion, or an extract, powdered agent, granules, tablets, or capsules, and a dispersing agent or stabilizer may be further included. Examples of the excipient may include lactose, fructose, sucrose, glucose, corn starch, starch, talc, sorbitol, crystalline cellulose, dextrin, kaolin, calcium carbonate, silicon dioxide, etc. Examples of the binder may include polyvinyl alcohol, polyvinyl ether, ethyl cellulose, methyl cellulose, gum Arabic, tragacanth, gelatin, shellac, hydroxypropyl cellulose, hydroxypropyl methylcellulose, calcium citrate, dextrin, pectin, etc. Examples of the glident may include magnesium stearate, talc, polyethylene glycol, silica, a cured plant oil, etc. For the colorant, anything whose addition in conventional pharmaceutical drugs is approved may be used. These tablets and granules may be appropriately coated with sugar coating, gelatin coating, etc., as necessary. Additionally, preservatives, antioxidants, etc. may be added as necessary.

According to an aspect of the present invention, there is provided a health functional food for improving cognitive function and memory containing osmotin as an active ingredient.

In the health functional food, the osmotin may be isolated from a plant and purified, and the plant may be one belonging to the genus Nicotiana. The osmotin may consist of an amino acid sequence represented by SEQ ID NO: 1, and it may be produced by genetic recombination technology using the cells of prokaryotes or eukaryotes such as yeasts, plants, insects, and mammals based on the above amino acid sequence.

According to another aspect of the present invention, there is provided a method of treating Alzheimer's disease in a subject, comprising administering to the subject a therapeutically effective amount of osmotin.

According to a further aspect of the present invention, there is provided a method for improving cognitive function and memory of a subject with Alzheimer's disease, comprising administering to the subject a therapeutically effective amount of osmotin.

### MODES FOR CARRYING OUT THE INVENTION

Hereinafter, the present invention will be explained in detail with reference to the following Examples. However, the present invention should not be limited by these Examples but can be performed in various mutually different forms. The following Examples are provided for the purposes of making the disclosure of the invention complete and fully informing one of ordinary skill in the art to which the present invention pertains the scope of the invention. Accordingly, the real technical protective scope of the present invention should be determined by the technical ideas of accompanying claims.

### Example 1: Measurement of Cognitive Function Through Behavioral Analysis of Animals

### 1-1: Experimental Animals

In the present invention, 8-week-old male C57BL/6J mice (WT) with a body weight of 23±1.5 g and transgenic C57BL/6J-Tg(NSE-APP_{SW})KLAR mice (hereinafter, abbreviated as ‘APPsw’) were purchased from the Jackson's laboratory (USA) and the Ministry of Food and Drug Safety (Republic of Korea), respectively. The breeding conditions provided for the mice were a light-dark cycle of day (16 hours)/night (8 hours) at 23° C. with humidity of 60±10%, and the mice were in ad libitum access to water and food.

1-2: Treatment with Osmotin

In the present invention, the osmotin, which was homogeneously purified from the previously-reported tobacco (Nicotiana tabacum cv. Wisconsin38) cells subjected to adaptive culture in 428 mM NaCl, was isolated to be used (Shah et al. Cell Death & Disease, 5: e1026, 2014). The osmotin was provided in a sterile solution (3 mg/mL, a ⅛-fold PBS) and the activity was measured at 0.2 μM, which is the IC₅₀ (the concentration of osmotin where the strain is reduced to 50% when treated with osmotin) to Saccharomyces cerevisiae strain BWG1-7a. Nine-month-old WT mice (30±1.2 g) and APPsw mice (39±0.8 g) were used. The osmotic was provided using sterile distilled water (vehicle) and intraperitoneally injected (15 mg/kg of body weight). For the molecular analysis of the effects of osmotin in brain tissue, each mouse was subjected to a behavior test, repeatedly injected with the vehicle or osmotin, and sacrificed after 12 hours.

### 1-3: Morris Water Maze Test

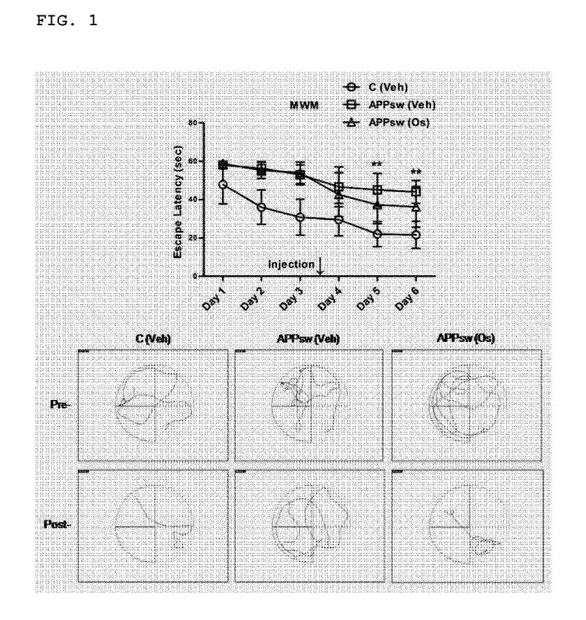

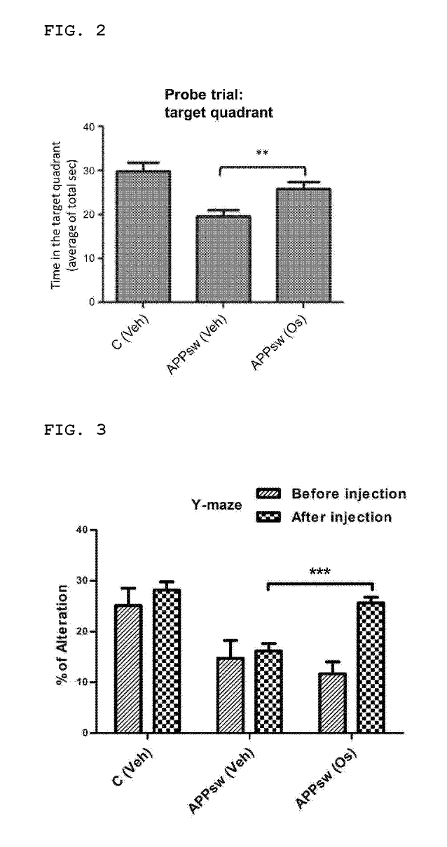

In the present invention, Morris Water Maze Test (MWM) was performed for the analysis of hippocampal-dependent learning including the learning of spatial memory and long-term memory. The test was performed as described below by applying a method disclosed previously (Morris, R. G. M., Learning and Motivation 12, 239-260, 1981). An experimental apparatus consisting of a circular water tank (diameter (10 cm)×height (40 cm)) and a platform for escape (diameter; 10 cm) was prepared and the water in the water tank (temperature; 22±1° C.) was filled up to a height of 26 cm. The hidden platform (diameter; 10 cm) was placed in a fixed position in the middle of a quadrant, about 1 cm below the water level, and a non-toxic white-aqueous dye was added to water to make it opaque and turbid. The water training was continuously performed for 6 days. In a water maze test, experimental animals are in search of the platform using the neighboring landmarks and thus the landmarks were retained constantly during the experimental period to avoid any environmental change. Each mouse was subjected to a training period twice daily, and each training consisted of two experiments so that the experiments can start from different directions of a quadrant. The standby time to escape from the water maze was calculated during the observation period of 60 seconds. When a mouse failed to find the platform in the water within 60 seconds, the mouse was smoothly guided to the platform and allowed to stay in the platform for 10 seconds to thereby remember the nearby clues again. The training was performed without the administration of osmotin or vehicle for the first 3 days, and 3 days after the training started, the dementia mouse model was administered with osmotin or vehicle only, and the normal mouse (control) was intraperitoneally injected with vehicle only, and the same training was repeated as described above. The platform was removed for a probe test. The time spent by each experimental animal was recorded on the quadrant of the platform. To examine the effects of osmotin on the spatial work memory, the investigation of change was first performed in an untreated mouse and then repeatedly performed in a dementia mouse model intraperitoneally injected with osmotin or vehicle only. The memory was measured by recording the standby time before escaping from the platform (FIG. 1). As a result, the escape standby time was gradually reduced for all the experimental groups, and on the 3^{rd} day, the dementia mouse model showed a significantly higher recordation of the escape standby time compared to that of the normal mouse (about a 1.8-fold). Even 3 days after the training started, the dementia mouse model was intraperitoneally injected with vehicle only or osmotin and the training was continued daily. The escape standby time gradually decreased in all of the groups over 6 days, however, the dementia mouse model treated with osmotin showed a higher rate of decrease compared to the dementia mouse model treated with vehicle only. Distinct differences in the escape standby time and length of exercise route were observed between the two groups on the 5^{th} day and the 6^{th} day. However, the average value of the escape standby time of the dementia mouse model treated with osmotin was still higher than that of the mouse treated with vehicle only, and this suggests that osmotin can partially recover spatial memory loss in an experimental condition. Then, the present inventors performed an MWM probe test on the 7^{th} day without a hidden platform (FIG. 2). The retention time in a quadrant including the conventional hidden platform was regarded as the scale of memory function. As a result, as illustrated in FIG. 2, the retention time was highest in the normal mouse treated with vehicle only (control), was at an intermediate level in the dementia mouse model administered with osmotin, and was significantly low in the dementia mouse model treated with vehicle only. These results indicate that osmotin can inhibit spatial memory loss in the hippocampus in the dementia mouse model.

### 1-4: Y-Maze Spontaneous Alternation Test

The present inventors examined the effect of osmotin on the spatial memory function in a transgenic dementia mouse model [APP_{SW}] using the Y-maze spontaneous alteration test. The Y-maze apparatus consisting of 3 arms, where each branch has a length of 50 cm, a width of 10 cm, and a height of 20 cm, and the branches are disposed at a 120° angle with each other, was used. Each mouse can explore the white Y-maze and each mouse was placed in the Y-maze and allowed to adapt to the environment before starting the behavior experiment. Then, the mouse was placed in the center of the Y-maze to face the junction of two arms toward a randomly selected direction, and was allowed to explore the maze for 8 minutes. The total number of entrances to each arm and continuous triplets were recorded by a behavior software system. The spontaneous alteration (%) was evaluated by measuring the number and the sequence of entrances into each arm. The alteration was acknowledged as 1 point when the mouse sequentially entered into three different arms (the actual alteration, i.e., when entered in the sequential order of ABC, BCA, and CAB). When the mouse did not enter continuously, no point was acknowledged. Accordingly, the alteration was calculated by the following equation:

Alteration (%)=[a set of 3 consecutive sequential entrance/a total number of entrances into arms−2]×100.

The alteration calculated as described above is regarded to be correlated with the spatial memory ability.

To examine the effect of osmotin on the spatial memory function, the Y-maze spontaneous alteration test was performed in the dementia mouse model administered with vehicle only. Then, the mouse was administered with osmotin or vehicle only, and the same experiment was repeated and the results were compared (FIG. 3). As illustrated in FIG. 3, the dementia mouse model showed a lower alteration compared to that of control, and this indicates a lower spatial memory. In the groups administered with vehicle only, both groups (the normal mouse as the control and the dementia mouse model) did not show any significant change with regard to alteration, whereas the group where the dementia mouse model was administered with osmotin showed a significant increase of alteration thus confirming that osmotin can alleviate the short-term memory impairment.

### Example 2: Analysis of Immunoassay

### 2-1: Western Blotting

Mouse hippocampus and cerebral cortex tissues in an amount of 10 mg, respectively, were extracted at 4° C. using the PRO-PREP (Intron, Korea) protein extraction solution (600 μL) according to the manufacturer's protocol. The protein concentrations were determined using the Bio-Rad Protein Assay Kit (Bio-Rad, USA). The lysates (proteins; 20 μg) were subjected to SDS-PAGE electrophoresis in 4% to 12% Bolt™ Mini Gels (Life Tech, USA), transferred onto nitrocellulose membranes, and blocked with 5% non-fat milk (or BSA). After reacting at 4° C. with primary antibody for at least 12 hours, the resultants were reacted with secondary antibody, to which horseradish peroxidase (HRP) was attached, and cross-reacting proteins were detected with ECL. The primary antibodies for PARP-1, phospho-CDK5 (Tyr 15), CDK5, SNAP-25, IDE, neprilysin/CD10(NEP), β-amyloid, BACE1, and phospho-tau (Ser⁴¹³) were purchased from Santa Cruz Biotech (USA) to be used, whereas the primary antibodies of β-actin, synaptophysin, phospho-AMPA R (Ser⁸⁴⁵), GABA_{B1}R, Calpain1, and phospho-CREB (Ser¹³³) were purchased from Cell Signaling Co., Ltd. (USA) to be used. The primary antibodies for amyloid precursor protein (APP) C-terminus, Nogo A, and Nogo-66 receptors were purchased from Millipore. The quantitative analysis for each band was performed using the Sigma Gel Software (SPSS, USA). The density values were calculated random unit (A.U.).

In the early stage of Alzheimer dementia, the decrease of cognitive memory is more closely associated with the loss of synapse density in the hippocampus than the appearance of Aβ plaques or NFT22, 23 (Scheff S W et al., Neurobiol. Aging., 27(10):1372-1384, 2006). As a result of western blotting analysis, it was confirmed that the expression levels of presynaptic vesicle membrane-specific proteins, synaptophysin, and SNAP-25 were significantly increased in APPsw mice when treated with osmotin (FIG. 4). Meanwhile, the phosphorylation of AMPA receptor subunit GluR1 at Ser⁸³¹ and Ser⁸⁴⁵ plays an important role in synaptic plasticity, and as a result of western blotting analysis, it was confirmed that the amount of the phosphorylated form at the Ser⁸⁴⁵ position of AMPA receptor subunit GluR1 was significantly increased in the dementia mouse model (APP_{SW}) treated with osmotin.

Not only the AMPA receptors but also Nogo receptors are known to play an important role in synaptic plasticity. The expression of GABABRs is controlled by the interaction between Nogo-66 receptor 1 (NgR1) and a ligand thereof, Nogo-(axon-inhibiting N-terminal domain of Nogo A). Accordingly, as a result of the analysis of the expression levels of NgR1, Nogo A, and GABA_{B1}R, the present inventors have confirmed that the expression of NgR1 and Nogo A significantly increased while the expression of GABA_{B1}R decreased in the hippocampus of the dementia mouse model (APPsw) compared to that of the control mouse, however, this phenomenon was recovered when treated with osmotin (FIG. 4). Phosphorylated-CREB produces a synapse, which enables a stronger resistance to the harmful effect of and activates the expression of genes associated with long-term improvement in learning and memory synaptic function in a model mouse with Alzheimer. Accordingly, the present inventors have also examined the expression feature of the phosphorylated CREB through the western blotting analysis, and as a result, have confirmed that the amount of phosphorylated-CREB (Ser¹³³) was further decreased in the APPsw mouse treated with vehicle only compared to that of the control, whereas the expression of phosphorylated-CREB (Ser¹³³) was increased (FIG. 4). Such a change in molecular level suggests that osmotin strengthens the synaptic plasticity, increases the regeneration of neuroaxons, and strengthens synaptic activity in the hippocampus of the Alzheimer model.

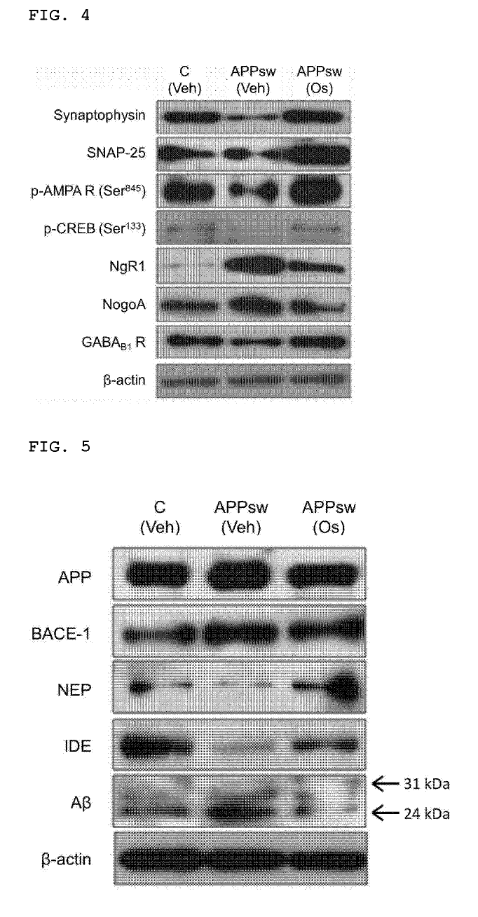

According to a previous report, synaptic dysfunction that causes the memory impairment in the brain with Alzheimer disease is caused by the accumulation of Aβ oligomers and generation of Aβ plaques (Saul et al., Neurobiology of Aging, 34(11), 2564-2573, 2013). Accordingly, Aβ oligomers and aggregates can become an early pathological marker for Alzheimer's disease in the brain tissue. As such, for the examination of the effect of osmotin on the accumulation of the Aβ oligomers and aggregates, the present inventors have analyzed the expression levels of amyloid precursor protein (APP) and Aβ peptides by western blotting analysis (FIG. 5). Through the results of FIG. 5, it was confirmed that the expression levels of amyloid precursor protein (APP) and Aβ peptides in the hippocampus of the APPsw mouse were higher than those in the control mouse, when treated with vehicle only.

Meanwhile, insulin degrading enzyme (IDE), which is a zinc metalloproteinase capable of decomposing Aβ₁₋₄₀, and neprilysin (NEP) have been strongly suggested that they can reverse the pathological hallmarks of Alzheimer disease. Accordingly, the present inventors have examined the expressions of IDE and NEP, in addition to Aβ peptides and amyloid precursor protein (APP), by western blotting analysis. As a result, they have confirmed that the expression levels of IDE and NEP were significantly low in the hippocampus of the dementia mouse model (APPsw) compared to the control mouse and significantly increased after the administration of osmotin. Furthermore, the level of beta-secretase 1 (BACE1), which is known to play an important role in the formation of Aβ peptides by protein cleavage, was also examined, and the expression of BACE1 was higher in the APPsw mouse, which was treated with vehicle only, whereas the expression of BACE1 was significantly reduced when treated with osmotin (FIG. 5).

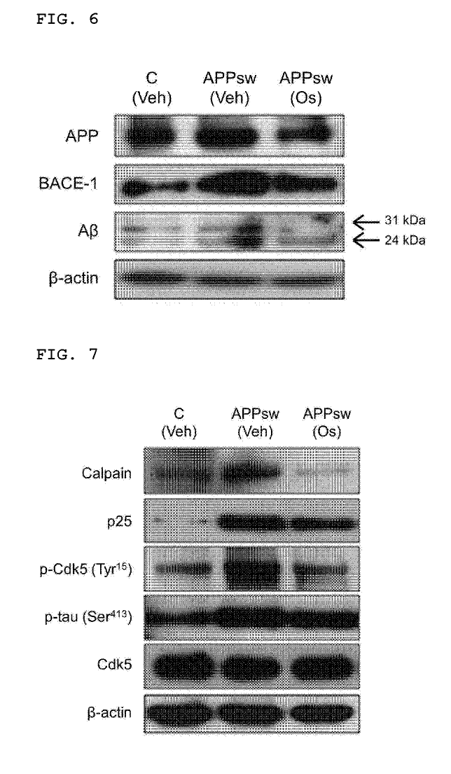

Additionally, to examine whether osmotin can inhibit the accumulation of Aβ peptides and Aβ plaques in cerebral cortex, western blotting analysis was performed with respect to cerebral cortex tissue (FIG. 6). As a result, it was confirmed that the amount of APP and/or Aβ peptides as well as Aβ plaques was increased in the prefrontal and piriform cortex of the APPsw mouse treated with vehicle only, compared to those of the control mouse which was treated with vehicle only, and the amount of Alzheimer markers was reduced in the APPsw mouse when treated with osmotin (FIG. 6).

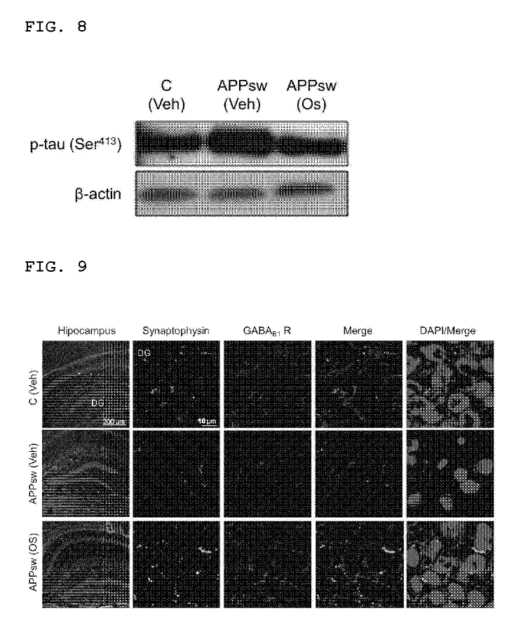

The phosphorylation in the S/T-P motif of tau, which is a microtubule-associated protein, is known to be the cause of the inclusion of tau in the paired helical filaments (PHF), which is discovered in the brain of Alzheimer patients (Noble et al., Neuron. 38(4):555-565, 2003). This pathological hallmark is a characteristic of neurons of Alzheimer patients and is known to be induced by Aβ peptides through the upregulation of the activity of Cdk5. In particular, Aβ peptides are known to induce calpain-mediated protein cleavage to p25, a stronger activator, in the Cdk5 activity of p35, which is a Cdk5-activating protein (Lee et al., Nature. 405(6784):360-364, 2000). In addition to the activation associated with p35/p25, Cdk5 is also activated by the phosphorylation of Tyr¹⁵. As such, the present inventors performed western blotting analysis with respect to calpain, p25, phosphorylated-Cdk5 (Tyr¹⁵), and phosphorylated-tau (Ser⁴¹³), which are marker proteins for Alzheimer disease, in the hippocampus of three experimental groups (the control mouse, the APPsw mouse administered with vehicle only, and the APPsw mouse administered with osmotin) (FIG. 7), and also performed western blotting analysis with respect to phosphorylated-tau using cerebral cortex tissues of the three experimental groups (FIG. 8). As a result, as illustrated in FIG. 7, the levels of calpain, p25, phosphorylated-Cdk5 (Tyr¹⁵), and phosphorylated-tau (Ser⁴¹³) was significantly increased in the hippocampus of the APP_{SW} administered with vehicle only compared to those of the control group, however, the expression of these markers was decreased in the APPsw mouse administered with osmotin. However, the expression of Cdk5 itself did not show any change and thus it was confirmed that these changes were caused by the regulation of phosphorylation of Cdk5 rather than by the change in the expression of Cdk5 itself. Meanwhile, in the results of the western blotting analysis with respect to cerebral cortex, the phosphorylated-tau (Ser⁴¹³) was shown to increase in the APPsw mouse administered with vehicle only compared to the control group, whereas the phosphorylated-tau (Ser⁴¹³) was shown to decrease in the APPsw mouse administered with osmotin (FIG. 8), thus confirming that osmotin can inhibit the expression levels of the molecular markers associated with Alzheimer's dementia disease in the cerebral cortex as well as in the hippocampus.

These results suggest that osmotin can alleviate the pathological hallmark of Alzheimer disease by not only removing the Aβ already formed in the APPsw mouse but also reducing the Aβ peptides from APP.

### 2-2: Tectological Analysis

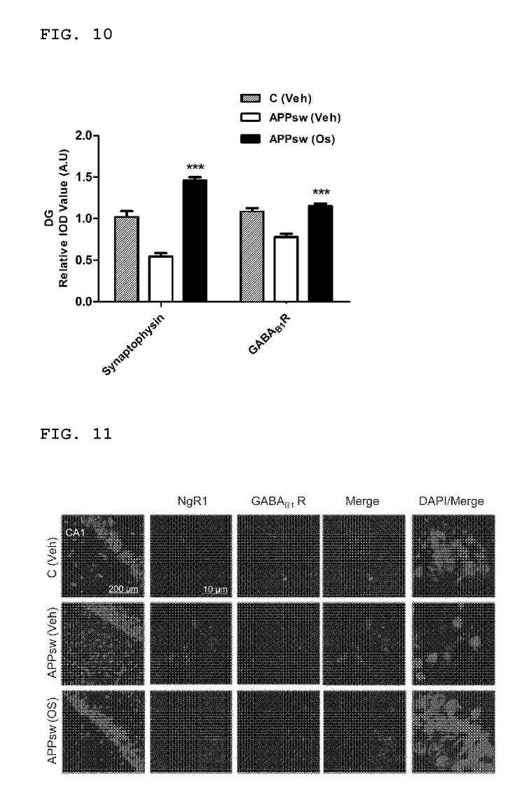

To examine whether the molecular level of changes in Examples observed by western blotting analysis can actually occur in brain tissues, tectological analysis was performed with respect to brains tissues of mice treated with osmotin. First, mice were transcardially fixed with ice-cold 4% paraformaldehyde. After collecting brains, they were fixed with 4% paraformaldehyde for 72 hours, transferred into 20% sucrose, and placed thereat. The brain tissues were embedded with an optimal cutting temperature compound (OCT) under a liquid nitrogen stream and cut into coronal sections using the CM 3050C cryostat (Leica) with a thickness of 14 μm. The tissue sections were mounted on the ProbeOn Plus slides (Fisher, USA). For the immunofluorescence analysis, the slides were washed twice with 0.01 M PBS for 15 minutes. After covering with a coverslip along with a proteinase K solution, each section was placed in a wet chamber at 37° C. for 5 minutes and then each section was placed in a PBS-blocking solution containing 5% normal goat serum and 0.3% Triton X-100 for 1 hour. After performing the blocking step, the slides were treated with primary antibody (diluted in a 1:100 ratio in the blocking solution) and reacted overnight. For the analysis, the primary antibodies to each of β-amyloid, phospho-tau (Ser⁴¹³), synaptophysin, GABA_{B1}R, and Nogo-66 receptors were used. After the reaction to the primary antibodies, immunocomplexes were reacted with secondary FITC-labeled antibody or secondary TRITC-labeled antibody (Santa Cruz Biotechnology, 1:50) for 1.5 hours and visualized. The slides were mounted with the Prolong Antifade reagent (Molecular Probes, USA) and examined under the confocal laser microscope (Flouview FV 1000). The thioflavine S staining with respect to amyloid plaques was performed after washing cryosections in a warm running tap water for a few minutes. The washed sections were soaked in 0.25% potassium permanganate for 5 minutes, soaked in 1% K₂S₂O₅ and 1% oxalic acid for 5 minutes, and then soaked in 0.02% thioflavine-S solution for 8 minutes. Then, the brain tissue sections were rinsed twice with 80% ethanol for 1 minute and washed slowly running tap water for 4 to 5 minutes. Then, the brain tissue sections were dehydrated by continuously soaking in 70%, 80%, and 95% alcohol and soaked in xylene. After mounting with coverslips, the tissue sections were examined under a confocal laser microscope (Flouview FV 1000). All the fluorescent signals were quantitated using the Image J Software (National Institutes of Health, USA) and indicated as integral optical density (IOD). As a result, it was confirmed that the expression of synaptophysin was increased in the dentate gyrus (DG) region of the hippocampus when administered with osmotin. Meanwhile, it was confirmed that the expression of GABA_{B1}R in the dentate gyrus (DG) region was also decreased in the APPsw mouse treated with vehicle only but the expression was recovered to that of the control when treated with osmotin (FIGS. 9 and 10).

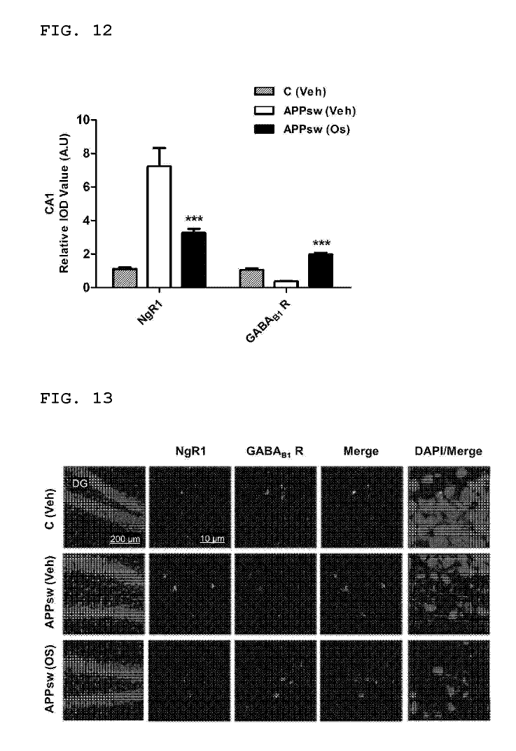

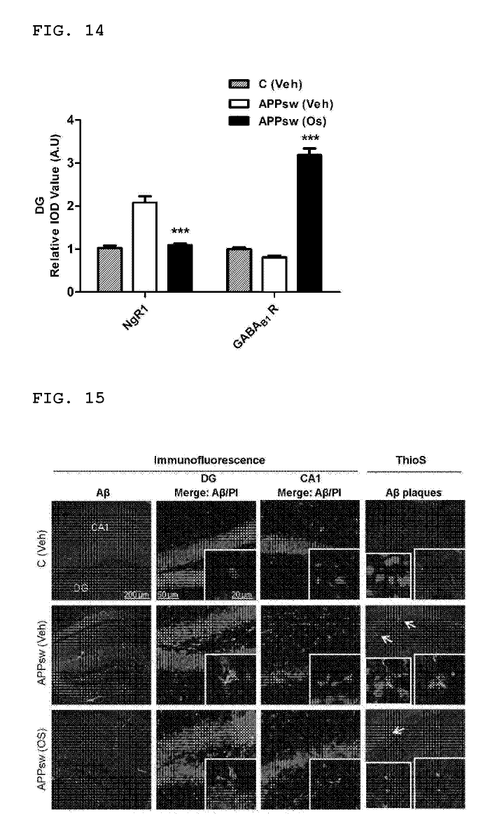

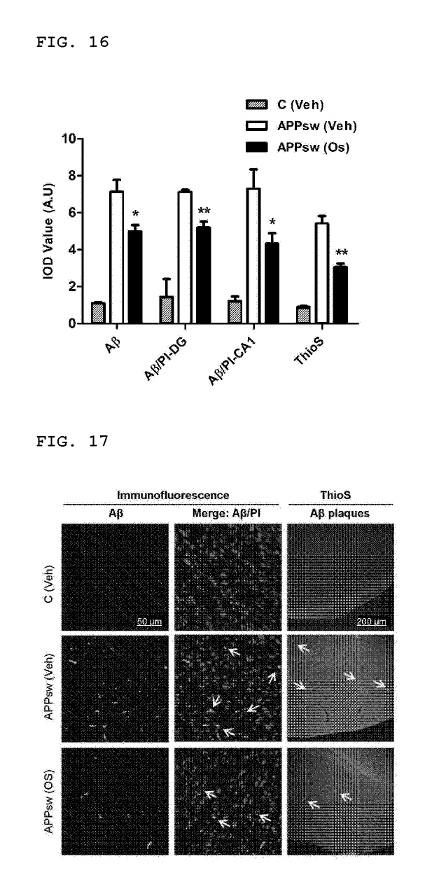

Nogo receptors are also known to play an important role in synaptic plasticity, and there was a report that the expression of GABABRs is partially regulated by the interaction between Nogo-66 receptor 1 (NgR1) and Nogo-66 (axon-inhibiting N-terminal domain of Nogo A), which is a ligand of NgR1. Accordingly, the present inventors have examined the expression of NgR1 in the Cornu Ammonis 1 (CA1) and the dentate gyrus (DG) regions of the hippocampus in the APPsw mouse, compared to that of the control mouse through the immunofluorescence analysis. As a result, overexpression of NgR1 in CA1 and DG regions of the hippocampus in the APPsw mouse compared to that of the control mouse was observed, and it was confirmed that the administration of osmotin to the APPsw mouse significantly reduced the expression level of NgR1. In particular, in the case of DG, the expression level of NgR1 was reduced to that of the control. Meanwhile, in the case of GABA_{B1}R, the result was shown to be the opposite to that of NgR1 (FIGS. 11 to 14).

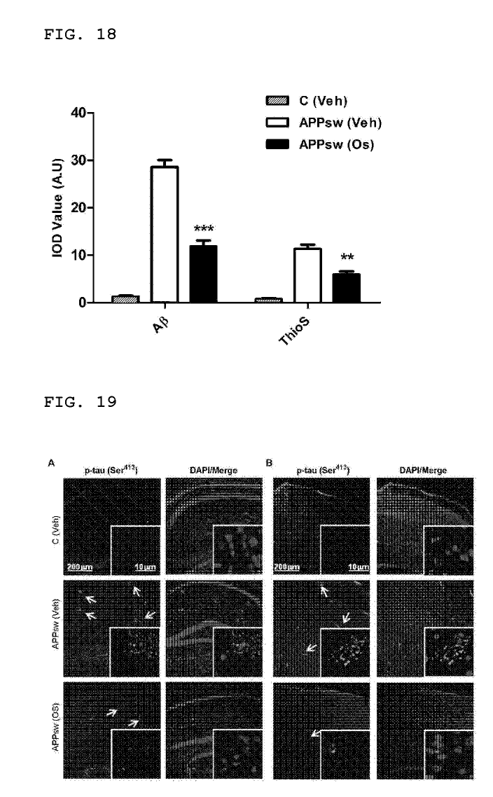

Additionally, the present inventors have performed an immunofluorescent assay on Aβ plaques, which are known to be a causative material for Alzheimer dementia, and thioflavine S staining, which is known to specifically stain Aβ (FIGS. 15 and 16). As a result, it was confirmed that Aβ peptides and Aβ plaques were almost not found in the hippocampus of the control mouse treated with vehicle only but they were mostly accumulated in the DG and CA1 regions of the hippocampus of the APPsw mouse treated with vehicle only. In the APPsw mouse treated with osmotin, the accumulation of total Aβ peptides and Aβ plaques was reduced in the DG and CA1 regions of the hippocampus compared to those of the APPsw mouse treated with vehicle only. Likewise, histochemical analysis was performed in the cerebral cortex (FIGS. 17 and 18) and the results obtained were similar to those of the western blotting analysis described above.

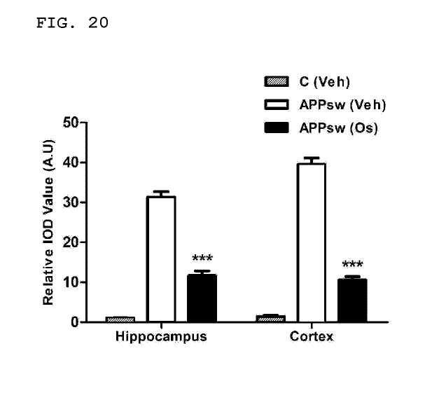

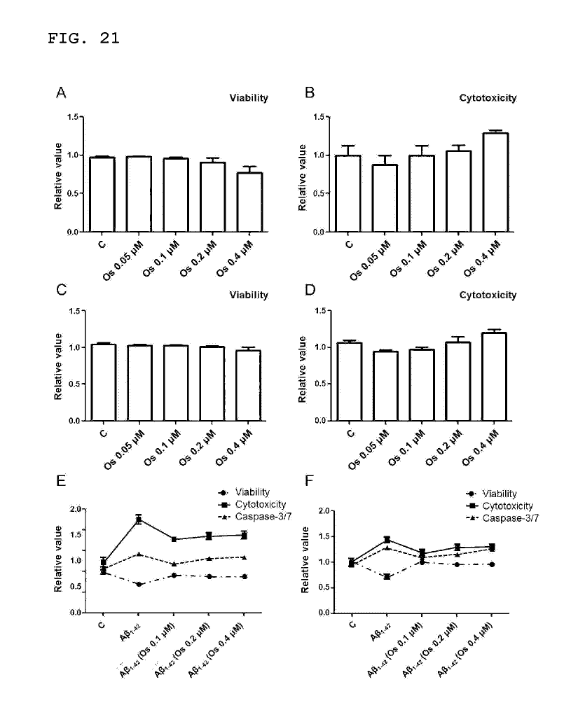

Furthermore, the present inventors have analyzed the amount of phosphorylated-tau (Ser⁴¹³) in the hippocampus and cerebral cortex by immunofluorescence analysis (FIGS. 19 and 20). FIG. 19A shows the staining result of p-tau (Ser⁴¹³) in the hippocampus and FIG. 19B shows the staining result of p-tau (Ser⁴¹³) in the cerebral cortex. FIG. 20 shows a graph quantitating the results of the immunofluorescent analysis. The results confirmed that when the APPsw mouse was administered with vehicle only the amount of p-tau (Ser⁴¹³) in the hippocampus and cerebral cortex was significantly increased compared to that of the control mouse, whereas the amount of p-tau (Ser′¹³) was significantly reduced when the APPsw mouse was administered with osmotin (FIGS. 19 and 20).

### Example 3: Viability, Cytotoxicity, and Caspase-3 Activity

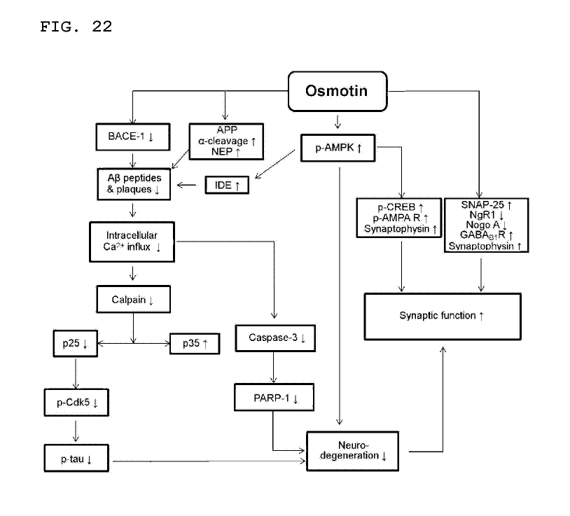

The present inventors obtained 17.5-day-old fetal brain tissue of rats from the gestational day (GD) in order to perform primary culture of neurons in the developing cerebral cortex and hippocampus. A preparative sample (100 μL) containing 2×10⁴ cells were aliquoted into two 96-well plates containing DMEM medium for cell growth containing 10% FBS and 1% penicillin-streptomycin, and cultured at 37° C., 5% CO₂ conditions. After 3 days, the medium was completely removed and 100 μL of a new growth medium was added to one of the plates (plate 1) while a growth medium containing 5 mM β-amyloid peptide (Aβ₁₋₄₂, Sigma, USA) was added to the other plate (plate 2). The plates were cultured for 24 hours, and then plate 1 was replaced with a medium supplemented with 0, 0.05, 0.1, 0.2, and 0.4 μg/mL of osmotin, whereas plate 2 was replaced with a growth medium containing Aβ₁₋₄₂ (5 mM) and 0, 0.1, 0.2, or 0.4 μg/mL of osmotin. After 24 hours, cell viability, cytotoxicity, and caspase-3/7 activity were measured using the ApoTox-Glo™ Triplex Assay (Promega, USA) kit and the Glomax® Multi Detection System (Promega, USA) according to the manufacturer's protocol. Osmotin, up to its concentration of 0.4 μM (10 μg/mL), did not show any significant effect on the viability or cytotoxicity of two different kinds of neurons in the hippocampus and cerebral cortex (FIGS. 21A to 21D). The cell viability accompanied in the cytotoxicity and caspase-3/7 activity was observed in the neurons of the hippocampus and cortical neurons when treated with Aβ₁₋₄₂ peptide (FIGS. 21E and 21F). When osmotin up to a maximum concentration of 0.4 μM was treated along with Aβ₁₋₄₂ peptide, the neurons of the hippocampus and cerebral cortex were protected from the harmful effect by Aβ₁₋₄₂ on cell viability, cytotoxicity, and caspase-3/7 activity (FIGS. 21E and 21F). The results revealed that, in the experimental concentration used in Examples in vitro, osmotin was non-toxic and protected neurons from the neural damage induced by Aβ, and these support the in vitro experimental results. Each of the data was indicated in means±SEM, and ANOVA analysis by Students' t-test was performed using the Prism 5 (GraphPad Software, Inc., San Diego, Calif., USA). The statistical significance was shown at P<0.05. FIG. 22 shows a flowchart summarizing the experimental results regarding by which route osmotin alleviates Alzheimer's disease. Osmotin is presumed to cause changes in APP processing, synaptic dysfuction, and neurofibrillary tangle through the activity of AMPK.

The present invention has been explained with reference to embodiments described above, however, they are provided only for illustrative purposes, and a skilled person in the art to which the present invention pertains will be able to understand that the present invention may be embodied in various modifications and other equivalent embodiments. Accordingly, the true technical scope of protection should be determined by the technical concepts of the appended claims.

### INDUSTRIAL APPLICABILITY

The osmotin according to an exemplary embodiment of the present invention was confirmed that it not only can pass through the blood brain barrier but also is effective for the improvement of cognitive function, thus being effectively used as a therapeutic agent derived from a natural substance for treating dementia.

### SEQUENCE LIST PRETEXT

SEQ ID NO: 1 is an amino acid sequence of osmotin derived from N. tabacum.

1-7. (canceled)

8. A method for improving cognitive function and memory of a subject with Alzheimer's disease, comprising administering to the subject a therapeutically effective amount of osmotin.

9. The method of claim 8, wherein the osmotin is isolated and purified from a plant belonging to the genus Nicotiana.

10. The method of claim 8, wherein the osmotin consists of an amino acid sequence represented by SEQ ID NO: 1.

11. The method of claim 8, wherein the osmotin is administered orally or parenterally.

12. The method of claim 11, wherein the osmotin is administered intravenously, subcutaneously, intramuscularly, or intraperitoneally.

13. The method of claim 8, wherein the osmotin is administered at a dose of 100 μg/kg to 1 mg/kg.

14. A method of treating or preventing Alzheimer's disease in a subject, comprising administering to the subject a therapeutically effective amount of osmotin.

15. The method of claim 14, wherein the osmotin is isolated and purified from a plant belonging to the genus Nicotiana.

16. The method of claim 14, wherein the osmotin consists of an amino acid sequence represented by SEQ ID NO: 1.

17. The method of claim 14, wherein the osmotin is administered orally or parenterally.

18. The method of claim 17, wherein the osmotin is administered intravenously, subcutaneously, intramuscularly, or intraperitoneally.

19. The method of claim 14, wherein the osmotin is administered at a dose of 100 μg/kg to 1 mg/kg.


SEQUENCE LISTING (1 sequence)

SEQ ID NO: 1 - protein, 243 aa, Nicotiana tabacum
MGNLRSSFVFFLLALVTYTYAATIEVRNNCPYTVWAASTPIGGGRRLDRGQTWVINAPRGTKMARVWGRTNCNFNAAGRGTCQTGDCGGVLQCTGWGKPPNTLAEYALDQFSGLDFWDISLLDGFNIPITFPTNPSGGKCHALCTAINGECPAELRVPGGCNNPCTTFGGQQYCCTQRPCGPTFFSKFFKQRCPDAYSYPQDDPTSTFTCPGGSTNYRVIFCPNGQAHPNFPLEMPGSDEVAK